(19) United States
(12) Patent Application Publication
(10) Pub. No.: US 2010/0055178 A1
(43) Pub. Date: Mar. 4, 2010

(54) ENTERIC-COATED CREATINE COMPOSITIONS AND METHODS OF USE THEREOF

(76) Inventor: Adel Vilallobos, Mission Hills, CA (US)

Correspondence Address: Nancy Lord, Ltd, 1970 N. LESLIE RD., NO. 220, PAHRUMP, NV, 89060, US

(21) Appl. No.: 12/201,007

(22) Filed: Aug. 29, 2008

(51) Int. Cl. A61K 9/48; A61K 9/00; A61K 9/28; A61K 9/16; A61K 31/197

(52) U.S. Cl. 424/463; 424/400; 514/565; 424/474; 424/490

(57) ABSTRACT

Compositions and methods are provided for supplementing dietary creatine and facilitating absorption of creatine, comprising an enteric-coated creatine pharmaceutical formulation that may be provided as a tablet, capsules, pellet, and microbead in the form of creatine monohydrate, creatine phosphate, creatine cytrate, creatine pyruvate, or creatine ethyl ester.

### FIELD OF THE INVENTION

The present invention relates to enteric-coated creatine supplementing dietary creatine and increasing strength, lean body mass and reducing body fat of an individual by orally administering an effective, lean body mass-increasing amount of a composition comprising an enteric-coated creatine.

### BACKGROUND OF THE INVENTION

The present invention relates to an oral creatine supplement, and the method for making this supplement. Taking creatine orally has been used to increase creatine and phosphocreatine stores in the human body. This is important for athletes because phosphocreatine increases the available energy to skeletal muscle.

When an athlete exercises or tenses a muscle, energy is required for the muscle to function properly. The energy it uses comes from several different sources, but primarily from nutrients obtained from food. These nutrients are broken down by natural processes occurring within the human body, and new compounds formed which are used to develop energy used by muscles. One of these compounds is adenosine triphosphate (ATP). When muscle energy is needed this ATP is broken down one step further into a chemical called adenosine diphosphate (ADP). This process releases energy which is then used by the contracting muscles. Without sufficient ATP, muscles do not perform properly.

Known energy increasers and stimulants have only superficially energized the body, and do not increase the body's ability to produce it's own ATP stores.

Muscle can store only limited amounts of ATP. As a result, it has been found that with about 5-10 seconds of muscle exertion, the amount of stored ATP is depleted. This results in muscle failure and fatigue. When this happens, the body tries to restore its immediate source of ATP by borrowing a high energy phosphate from a chemical called phosphocreatine (PCr). Muscle cells store the chemical, PCr, in the same way it stores ATP. If high intensity exercise goes beyond 10 seconds, the body will continue to try and restore its ATP levels by a process called glycolysis. This process is complicated and is a slow method of restoring ATP levels. This is a special problem for anaerobic athletes who require instant energy to maintain and sustain high powered muscle contractions.

By orally supplementing with creatine, an athlete can enhance his body's storage levels of PCr As the muscle runs out of ATP, it can recharge itself by borrowing a phopshate group from the PCr molecule. Research has shown that by supplementing with 5 grams of creatine, 4-6 times a day, for two or more days, the human body showed a significant increase in total creatine concentration.

ATP or PCr cannot be ingested directly by athletes because these chemicals are destroyed by the digestive system of the athlete. However, it has been found that creatine can be ingested and converted by the body to PCr. The resulting cellular concentrations of creatine after administration, is stable and is not prone to dissipation.

The most commonly used and most researched oral creatine supplement is creatine monohydrate. The most commonly used amounts have varied from 500 mg to 10 grams daily. It has been taken in powder, capsule, tablet, and liquid form. The creatine is mixed with or taken with water, fruit juice, acidic effervescent drink or acidic fruit flavored drinks.

Other than creatine monohydrate, other forms of creatine have also been used, such as creatine citrate, creatine phosphate, creatine ethyl ester, and creatine pyruvate. These other forms of creatine are administered similar to the method of administrating creatine monohydrate.

The main problem with all existing creatine supplementation is the ability to deliver creatine in a usable form by the human body. Current delivery systems do not protect the degradation of creatine, which breaks down rapidly to creatinine in acidic environments like the stomach. Creatinine is a toxic byproduct of creatine. It is believed that the main reason for complaints resulting from creatine consumption, namely, stomach cramps, edema, and dehydration, are caused by the body's defense to this toxic compound.

The known oral creatine supplements are dissolved in acidic solutions having a pH range from 3-6 and more importantly, the pH of the stomach is even lower. Research has shown that at these pH levels, the rate of conversion of creatine to creatinine is almost instantaneous.

From the above, it may be ascertained that a need exists for a method of enhancing the delivery of usable creatine to humans without substantial creatinine being formed. Further, a need exists for an oral creatine supplement that is in the form of a capsule, tablet, pellet or microbead that is enteric coated to insure efficient delivery to the body and muscles.

### SUMMARY OF THE INVENTION

One embodiment of the present invention is a composition for supplementing dietary creatine and facilitating absorption of creatine, the composition comprising an enteric-coated creatine formulation. Advantageously, the formulation is a tablet, capsule, pellet, or microbead. Specifically, the microbead is a sugar beadlet or microcrystalline cellulose beadlet.

The present invention also provides a method for supplementing dietary creatine in an individual, comprising orally administering to the individual a composition comprising an enteric-coated creatine formulation. In one aspect of this preferred embodiment, the formulation is a tablet, capsule, pellet, or microbead. Specifically, the microbead is a sugar beadlet or microcrystalline cellulose beadlet.

Another embodiment of the invention is a method for increasing lean body mass and reducing body fat of an individual in need thereof, comprising orally administering to the individual an effective, lean body mass-increasing amount of a composition comprising an enteric-coated creatine formulation. In one aspect of this preferred embodiment, the formulation is a tablet, capsule, pellet or microbead. Preferably, the microbead is a sugar beadlet or microcrystalline cellulose beadlet and the creatine is coated on the beadlet.

The present invention also provides a method for reducing high levels of blood serum lipids in an individual in need thereof, comprising administering to the individual an effective blood serum lipid-reducing amount of a composition comprising an enteric-coated creatine formulation. In one aspect of this preferred embodiment, the formulation is a tablet, capsule, pellet or microbead. Preferably, the microbead is a sugar beadlet or microcrystalline cellulose beadlet and the creatine is coated on the beadlet.

### DETAILED DESCRIPTION OF THE PREFERRED EMBODIMENTS

The present invention provides enteric-coated compositions comprising an enteric-coated creatine formulation. Any pharmaceutical formulation well known in the art can be coated with an enteric coating. In a specific embodiment, the formulation is a tablet, capsule, pellet or microbead. Also described are methods for supplementing dietary creatine and increasing strength and lean body mass, and reducing body fat by the administration of the enteric-coated creatine.

For oral administration, the creatine monohydrate, creatine phosphate, creatine ethyl ester, creatine pyruvate, creatine citrate, or other forms of creatine may be incorporated into a tablet, capsule, pellet or microbead. Compositions may be prepared according to any method known in the art for the manufacture of pharmaceutically acceptable compositions and such compositions may contain one or more of the following agents: sweeteners, flavoring agents, coloring agents and preservatives. Tablets containing the active ingredients in admixture with non-toxic pharmaceutically acceptable excipients suitable for tablet manufacture are acceptable. “Pharmaceutically acceptable” means that the agent should be acceptable in the sense of being compatible with the other ingredients of the formulation (as well as non-injurious to the individual). Such excipients include inert diluents such as calcium carbonate, sodium carbonate, lactose, calcium phosphate or sodium phosphate; granulating and disintegrating agents, such as corn starch and alginic acid; binding agents such as starch, gelatin or acacia; and lubricating agents such as magnesium stearate, stearic acid or talc. Tablets may be uncoated or may be coated with known techniques to delay disintegration and absorption in the gastrointestinal tract and thereby provide a sustained action over a longer period of time. For example, a time delay material such as glyceryl monostearate or glyceryl stearate alone or with a wax may be employed.

The enteric coating prevents dissolution of the tablet, capsule, pellet or microbead in the acidic environment of the stomach. Instead, this coating dissolves in the small intestine at a more neutral pH. Because creatine might be more stable at this neutral pH than at the acidic pH of the stomach, enhanced absorption occurs because the creatine remains substantially intact until it reaches the small intestine. Such enteric coated compositions are described by Bauer et al., Coated Pharmaceutical Dosage Forms. Fundamentals, Manufacturing Techniques, Biopharmaceutical Aspects, Test Methods and Raw Materials, CRC Press, Washington, D.C., 1998, the entire contents of which are hereby incorporated by reference. Specific suitable coatings are available as Opadry Enterics by Colorcon of West Point, Pa.; and the EUDRAGIT polymers of Rohm Pharma, GmbH.

Formulations for oral use may also be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, such as peanut oil, liquid paraffin or olive oil.

Conventional tablet mixing and compression techniques, and related equipment, known to those of skill in the art may be used. Tablet mixing and compression methods that may be used with the present invention include wet granulation, fluid bed granulation, dry granulation or direct compression. See Remington's Pharmaceutical Sciences, Ch. 89, Mack Publishing (18th Ed. 1990). In addition, tableting equipment and machinery, including single punch machines, and rotary tablet machines, including high speed and multi-layer equipment, can be used, again as described in Remington's, supra.

In a specific embodiment, the creatine monohydrate, creatine phosphate, creatine citrate, creatine ethyl ester, or creatine pyruvate is coated onto a microbead. In a preferred embodiment, these microbeads are sugar beadlets of various sizes, also known as nonpareils, and are commercially available from, for example, SmithKline Beecham. If the microbeads are to be used to administer the compositions of the invention to diabetic patients, the administration of other types of microbeads, such as microcrystalline cellulose, is preferred. Microcrystalline cellulose is commercially available and can be processed into beadlets of various sizes by micronization, a technique well known in the art. The microbeads are essentially a carrier for the compositions of the invention. For a description of coated beadlets, see, for example, Carstensen, J. T., Pharmaceutical Principles of solid Dosage Forms, Technonic Publishing Co., Inc., Lancaster, Pa., pp. 228-230, 1993, hereby incorporated by reference. Aqueous solutions containing the creatine components of the composition are sprayed onto the microbeads by well known methods, by suspending the microbeads in an upcurrent of air and introducing a fine spray of the active ingredients which form a coating on the outside of the microbeads which is then allowed to dry. Optionally, the coated microbeads can be further coated with a substance to protect the active ingredients coated onto the beads, such as latex. The microbeads may be placed in a capsule prior to administration. In another specific embodiment, the capsule or the microbeads are coated with an enteric coating to delay dissolution until reaching the small intestine.

Coating may be performed by any one of numerous processes known to those of ordinary skill in the art. U.S. Pat. No. 6,139,875, to Adams, describes a method an aqueous enteric coating composition of a dispersion of about 5 to 45 weight percent of a hydrophobic compound having from about 12 to 20 carbon atoms selected from the group consisting of alcohols, fatty acids and salts thereof, fatty amides and salts thereof, and lipids and about 5 to 50 weight percent of a water-insoluble flake material, dispersed in an aqueous solution of about 35 to 70 weight percent of a water-soluble salt of an enteric polymer selected from the group consisting of cellulose acetate phthalate (C-A-P), cellulose acetate trimellitate (C-A-T), hydroxypropyl methylcellulose phthalate (HPMCP), methacrylic acid/ethyl acrylate copolymer, hydroxypropyl methylcellulose acetate succinate (HPMCAS), and polyvinyl acetate phthalate (PVAP), wherein the weight percentages are based on a total solids weight, and is incorporated by reference herein.

Patent Cooperation Treaty application No. PCT/US1997/019296, to Stephen Wu, teaches an enteric coating composition comprising a blend of a) an alkali-soluble acrylic latex polymer and b) an aqueous solution of ammonium or alkaline salts of cellulose polymers that may be used in this invention and is incorporated by reference herein. The coating compositions of Wu are insoluble in a low pH environment such as the stomach, where the fluid has a pH value normally less than about 3.5, but dissolve rapidly or swell sufficiently to disintegrate in a high pH environment, such as intestinal fluid, which has a pH value normally greater than about 5.0.

During blending, the acrylic latex particles are softened, and intimately associated with and surrounded by ammonium or alkaline salts of cellulosic enteric polymers in an aqueous medium. Thus, the ammonium or alkaline salts of cellulosic enteric polymers serve as a protective colloid for the latex particles so that the blend exhibits unique properties suitable for aqueous enteric coating applications. The resulting blend is much less sensitive to heat and high shearing forces than the acrylic latex alone.

The novel enteric coating method of Obara may also be used. This method involves direct feeding of coating polymer powder and simultaneous spraying of plasticizing agent, without either organic solvent or water, using a centrifugal granulator, fluidized bed, or tablet-coating machine. For film formation, a curing step was then necessary; this involved spraying a small amount (3-8% of core weight) of water or hydroxypropyl methylcellulose solution, followed by heating. Hydroxypropyl methylcellulose acetate succinate was used as the enteric coating polymer, and a combination of triethyl citrate and acetylated monoglyceride was used for plasticization. The coated beads and tablets were evaluated for gastric resistance, intestinal disintegration, and stability, in comparison with beads and tablets from a conventional aqueous coating with the same enteric polymer. The new method required a higher coating amount for gastric resistance compared with the conventional coating, but the processing time was dramatically reduced. The results show that this dry coating method is applicable to the preparation of enteric-coated beads and tablets using commercially available lab-scale apparatus. Obara, S, et al, 47(1) European journal of pharmaceutics and biopharmaceutics 51-55 (1999).

Alternatively, the method described by Felton, Surtevant and Birkmire of GEA Niro Pharma Systems, may be employed. Capsules are rapidly rotated as they pass continuously through a coating zone, potentially allowing for very uniform film formation on all surfaces of the capsules. A summary of the poster presented at the 2006 AAPS Annual Meeting & Exposition, October 2006 in San Antonio, Tex., and their Supercell™ Coating Technology (SCT) and apparatus is described in U.S. Pat. No. 6,209,479 and EP patent EP 1,140,366, all of which are incorporated by reference herein. This technology offers the ability to uniformly and rapidly coat small batches of objects having a major diameter between 3 to 35 mm with a high degree of accuracy. The coater consists of a conical processing chamber that sits on top of a gas distribution plate (roto-nozzle). The roto-nozzle contains gas jets designed to accelerate the objects for coating, e.g. capsules, through the coating zone in a ballistic flight path. The gas jets impart momentum to the capsules such that they are rotating as they pass through the coating zone (FIG. 3). Coating material is atomized by means of a specially designed low-momentum two-fluid spray nozzle located below the gas distribution plate (FIGS. 4 and 5). Normally, the momentum from a two fluid nozzle can accelerate objects such as capsules fast enough that they are easily damaged when they strike a surface or fall. In SCT, the atomizing gas is mixed with low pressure drying gas in order to dissipate the momentum generated in the atomizing process. Capsules are coated co-currently with the drying gas. At the end of the coating process, the capsules are pneumatically conveyed out of the processing chamber.

EUDRAGIT polymers, and most specifically, the EUDRAGIT L100 and LS 100 Polymers of Rohm Pharma, GmbH may be employed in coating the microbeads, tablets, or capsules. For the processing of EUDRAGIT usually some additional excipients are required. Plasticizers reduce the minimum film forming temperatures as well as the glass transition temperatures. The addition of a plasticizer like triethyl citrate increases the flexibility of the film coatings. To prevent the film coatings from getting sticky some glidants such as talc or glycerol monostearate are also necessary. EUDRAGIT L 100 is pH dependent anionic polymer powder solubilizing above pH 6.0 for targeted drug delivery in the jejunum and EUDRAGIT L S 100 is pH dependent anionic polymer powder solubilizing above pH 7.0 for targeted drug delivery in the jejunum.

To process using a EUDRAGIT polymer, the solution is pre-warmed to about 30° C., by means of an air spray gun, the spray rate adjusted for inlet air quantity and inlet air temperature such a way that spraying can be performed continuously. The tablet-bed temperature is adjusted to approximately 25 to 35 Â ° C. during the process. The solution is sprayed at low rates initially for moisture-sensitive tablets (seal-coating), and at faster rates after the 30 minute-sealing coating. If twinning occurs, spray is stopped and pan speed increased. After coating, the tablets are dried by a slowing of the pan speed.

Typically, the dosage range of creatine administered to an individual in the form of creatine monohydrate, creatine phosphate, creatine citrate, creatine pyruvate, or creatine ethyl ester provides between about 500 mg and about 10 gm of creatine; more specifically between about 1000 mg and about 5000 mg per day; most specifically, about 2000 mg per day.

The composition is provided for supplementing dietary creatine and facilitating absorption of creatine, the composition comprising an enteric-coated creatine pharmaceutical formulation and may be provided as a tablet, capsule, pellet, and microbead.

In another embodiment of the invention, methods are provided for supplementing dietary creatine in an individual, comprising orally administering to an individual a composition comprising an enteric-coated creatine, more specifically in the form of a tablet, capsule, pellet and microbead. The creatine may be provided as creatine monohydrate, creatine phosphate, creatine cytrate, creatine pyruvate, or creatine ethyl ester. Dosage may range from between about 500 mg and about 10 gm of creatine; more specifically between about 1000 mg and about 5000 mg per day; most specifically, about 2000 mg per day.

Also provided are methods for increasing strength and lean body mass, and reducing body fat of an individual in need thereof, comprising orally administering to an individual an effective, lean body mass-increasing amount of a composition comprising an enteric-coated creatine, more specifically in the form of a tablet, capsule, pellet and microbead. The creatine may be provided as creatine monohydrate, creatine phosphate, creatine cytrate, creatine pyruvate, or creatine ethyl ester. Dosage may range from between about 500 mg and about 10 gm of creatine; more specifically between about 1000 mg and about 5000 mg per day; most specifically, about 2000 mg per day.

### Example 1

One kg creatine monohydrate pellets, about 1 mm in core size are coated using EUDRAGIT FS 30 D. 210 g water is heated to 70° C. During homogenization, 10 grams of triethyl citrate and 10 grams of polysorbate 80 are added, then about 8 grams of glycerol monostearate is added and homogenized in this mixture for 10-20 minutes. Then, 250 grams (ml) of water is then poured into the suspension, which is stirred with conventional stirrers until the temperature is below 30° C. Pigment may be added. Finally, the Glycerol monostearate suspension is slowly poured into a 667 EUDRAGIT® dispersion with gentle stirring. The spray suspension is poured through a 0.5 mm sieve and stir during the coating process to prevent the solids from settling. With decreasing volume of the spray suspension, the stirring speed is be adjusted in order to avoid foam formation.

To coat the pellets, the pellets are fluidized in the equipment by adjusting the desired air supply, the inlet air temperature (35-40° C.) and the atomizing air (2 bar). After a 5 minute warm-up time of about 5 minutes, the peristaltic pump is adjusted to approximately 8-12 grams of coating suspension per minute and is sprayed continuously onto the tumbling pellets. During the spraying process the outlet air temperature decreases to about 23-25° C. The pellets must float freely. In case of agglomeration, interrupt the spraying process, the pellets are dried until they tumble freely again, then continue spraying at slightly reduced speed. The spray application takes about 106 minutes altogether. Subsequently, dry the coated material for 5 minutes with reduced tumbling, less whirling action, spread it on trays and dried an airflow oven at 40° C. for approximately 2 hours.

### Example 2

Ten bodybuilders ingest 2000 mg per day of microbeads enteric-coated with EUDRAGIT LS 100 for six (6) weeks. Prior to and at the end of the study, weight and lean body mass are measured. During the study, the bodybuilders maintain their usual exercise regimen. At the end of the trial, 8 of the 10 bodybuilders lose between 4 and 17 lbs, and lean body mass is increased to a statistically significant amount (P<0.05).

While this invention has been particularly shown and described with references to preferred embodiments thereof, it will be understood by those skilled in the art that various changes in form and details may be made therein without departing from the spirit and scope of the invention as defined by the appended claims. Those skilled in the art will recognize or be able to ascertain using no more than routine experimentation, many equivalents to the specific embodiments of the invention described specifically herein. Such equivalents are intended to be encompassed in the scope of the claims.

What is claimed is:

1. A composition for supplementing dietary creatine and facilitating absorption of creatine, said composition comprising an enteric-coated creatine pharmaceutical formulation.

2. The composition of claim 1, wherein said enteric-coated creatine is selected from the group consisting of a tablet, capsule pellet, and microbead.

3. The composition of claim 1 wherein said creatine is comprised of creatine monohydrate.

4. The composition of claim 1 wherein said creatine is comprised of creatine phosphate.

5. The composition of claim 1 wherein said creatine is comprised of creatine pyruvate.

6. The composition of claim 1 wherein said creatine is comprised of creatine cytrate.

7. The composition of claim 1 wherein said creatine is comprised of creatine ethyl ester

8. A method for supplementing dietary creatine in an individual, comprising orally administering to said individual a composition comprising an enteric-coated creatine.

9. The method of claim 8, wherein said enteric coated creatine is selected from the group consisting of a tablet, capsule, pellet and microbead.

10. The method of claim 9 wherein said enteric coated creatine is comprised of creatine monohydrate.

11. The method of claim 9 wherein said enteric coated creatine is comprised of creatine phosphate.

12. The method of claim 9 wherein said enteric coated creatine is comprised of creatine pyruvate.

13. The method of claim 9 wherein said enteric coated creatine is comprised of creatine citrate.

14. The method of claim 9, wherein said creatine is administered in an amount ranging from about 500 mg to about grams per day.

15. The method of claim 13, wherein said creatine is administered in an amount of about 2000 mg per day.

16. A method for increasing lean body mass and reducing body fat of an individual in need thereof, comprising orally administering to said individual an effective, lean body mass-increasing amount of a composition comprising an enteric-coated creatine.

17. The method of claim 16, wherein said pharmaceutical formulation is selected from the group consisting of a tablet, capsule and microbead.

18. The method of claim 17, wherein said creatine is administered in an amount ranging from about 500 mg to about grams per day.

19. The method of claim 18, wherein said creatine is administered in an amount of about 2000 mg per day.

20. A method for increasing strength of an individual in need thereof, comprising orally administering to said individual an effective, strength increasing amount of a composition comprising an enteric-coated creatine.
